(19) United States
(12) Patent Application Publication
(10) Pub. No.: US 2026/0240433 A1
(43) Pub. Date: Aug. 20, 2026

(54) RETINAL FLUORESCENCE IMAGING SYSTEM

(71) Applicant: Aristomenis Thanos, Portland, OR (US)

(72) Inventor: Aristomenis Thanos, Portland, OR (US)

(21) Appl. No.: 19/543,580

(22) Filed: Feb. 18, 2026

Related U.S. Application Data
(60) Provisional application No. 63/759,776, filed on Feb. 18, 2025.

(51) Int. Cl. A61B 3/12; A61B 5/00

(52) U.S. Cl. CPC A61B 3/1241; A61B 5/0071

(57) ABSTRACT

A retinal fluorescence imaging system and method for diagnosing and managing ocular diseases are disclosed. Fluorophore-tagged biomolecules, such as antibodies or antibody fragments, are intraocularly administered to bind pre-specified targets within the retina or choroid. Excitation and emission are captured by an ocular imaging device, including confocal scanning laser ophthalmoscope or fundus camera, enabling real-time detection of anatomical or pathological targets. The system supports co-administration of multiple biomolecules with distinct fluorophores for simultaneous disease detection and includes shielding to minimize photobleaching.

### FIELD OF INVENTION

The present disclosure pertains to optical imaging technologies, specifically to non-invasive in vivo retinal fluorescence imaging systems for the diagnosis and management of ocular diseases.

### BACKGROUND

Fluorescence is a type of photoluminescence that occurs when susceptible molecules known as fluorophores absorb electromagnetic energy, temporarily exciting them to a higher energy state. As the molecules return to their original energy level, they emit light of a different, usually longer wavelength.

Fluorescein and indocyanine green have been the prototypical fluorescent dyes used in ophthalmology. These dyes are most commonly injected intravenously and circulate within the ocular blood vessels. Their use has been instrumental in the evaluation and management of retinal diseases.

In the case of fluorescein performed by standard fundus camera, blue light is used to illuminate the retina and excite the fluorescein, and barrier filters allow only yellow-green light (from the fluorescence) to reach the camera. Alternatively, stimulation of these dyes is achieved using various lasers of different wavelengths in the case of confocal scanning laser ophthalmoscopes.

Confocal scanning laser ophthalmoscopes (cSLO) are used in ophthalmic clinics routinely for retina imaging and fluorescence angiography as a safe imaging modality. Specifically, a cSLO uses visible or near-IR light sources to continuously scan the retina surface spot by spot. Compared with traditional ophthalmoscopes, cSLOs have the advantages of low radiation, high contrast, high collection efficiency, and mydriasis-free imaging. The confocal feature of cSLOs ensures only the light returned from the focused plane in the object space is collected, thus greatly improving the contrast of the images.

However, beyond the diagnostic capabilities of fluorescein and indocyanine green, the use of fluorescence in retinal diseases is limited. Often, the diagnosis of inflammatory retinal conditions rely on the performance of laboratory diagnostic tests, and obtaining intraocular tissue samples (aqueous and/or vitreous humor). In addition, due to the size of the eye, the samples are falsely negative and have very low yield.

Disease diagnosis is often delayed due to limited diagnostic value of obtained intraocular samples. Frequently, in some diseases, patients need to undergo multiple diagnostic surgeries for the diagnosis to be established, like in the case of intraocular lymphoma.

### SUMMARY

In one general aspect, method may include intraocularly administering to the patient a fluorophore-tagged biomolecule that specifically binds a pre-specified target within a retina or a choroid of the patient. Method may also include exciting the fluorophore in vivo and capturing emitted light from the biomolecule with an ocular imaging device. Method may furthermore include determining a presence, distribution, or extent of the target from the captured emission. Other embodiments of this aspect include corresponding computer systems, apparatus, and computer programs recorded on one or more computer storage devices, each configured to perform the actions of the methods.

Implementations may include one or more of the following features. Method where the biomolecule may include an antibody or an antibody fragment. Method where the ocular imaging device may include a confocal scanning laser ophthalmoscope or a fundus camera. Method may include shielding the eye after the administering to reduce photobleaching. Method where the administering may include co-administering multiple biomolecules, each conjugated to a different fluorophore. Method where the target may include an internal limiting membrane of the retina and the determining may include delineating peeled versus unpeeled regions for pre-operative planning. Method where the target may include lymphoma cells infiltrating the retina. Method where the target may include an infectious microorganism present in the retina. Method where the capturing is performed approximately 1-2 hours after the administering. Method where exciting the fluorophore and capturing the emitted light may include selecting an excitation wavelength matched to an absorption band of the fluorophore and filtering detected light with at least one barrier filter configured to preferentially pass an emission band of the fluorophore while attenuating the excitation wavelength. Implementations of the described techniques may include hardware, a method or process, or a computer tangible medium.

In one general aspect, retinal fluorescence imaging system may include at least one fluorophore-tagged biomolecule configured for intraocular administration and specific binding to a pre-specified target within a retina or a choroid of the patient. Retinal fluorescence imaging system may also include an ocular imaging device configured to excite the fluorophore in vivo and capture emitted light from the biomolecule. System may furthermore include a processor configured to determine a presence, distribution, or extent of the target from the captured emission. Other embodiments of this aspect include corresponding computer systems, apparatus, and computer programs recorded on one or more computer storage devices, each configured to perform the actions of the methods.

Implementations may include one or more of the following features. System where the biomolecule may include an antibody or an antibody fragment. System where the ocular imaging device may include a confocal scanning laser ophthalmoscope or a fundus camera. System may include a shielding element configured to cover the eye after administration to reduce photobleaching. System where the system is configured to co-administer multiple biomolecules, each conjugated to a different fluorophore. System where the target may include an internal limiting membrane of the retina and the processor is configured to delineate peeled versus unpeeled regions for pre-operative planning. System where the target may include lymphoma cells infiltrating the retina. System where the target may include an infectious microorganism present in the retina. System where the ocular imaging device is configured to capture emission approximately 1-2 hours after administration. Implementations of the described techniques may include hardware, a method or process, or a computer tangible medium.

In one general aspect, non-transitory computer-readable medium storing instructions may include receiving image data representing captured fluorescence emission from fluorophore-tagged biomolecules within a retina or choroid of a human patient. Non-transitory computer-readable medium storing instructions may also include analyzing the captured fluorescence emission to determine a presence, distribution, or extent of a pre-specified target within the retina or choroid. Instructions may furthermore include processing the image data using one or more algorithms having at least one of signal amplification, noise reduction, or image enhancement. Other embodiments of this aspect include corresponding computer systems, apparatus, and computer programs recorded on one or more computer storage devices, each configured to perform the actions of the methods.

### BRIEF DESCRIPTION OF DRAWINGS

FIG. 1A illustrates the structural representation of biomolecules, including full-length antibodies and their fragments.

FIG. 1B illustrates biomolecules conjugated to fluorophores.

FIG. 2A illustrates the interaction and binding of fluorophore-conjugated biomolecules with retinal tissue, such as the internal limiting membrane.

FIG. 2B illustrates the stimulation of fluorophore-conjugated biomolecules bound to retinal layers using electromagnetic wavelengths for diagnostic or therapeutic purposes.

FIG. 2C illustrates the capture of emitted wavelengths from fluorophore-tagged biomolecules within retinal layers using a specific camera or cSLO for diagnostic purposes.

FIG. 3 depicts a microscopy image of a non-human primate retina labeled with a fluorophore-conjugated antibody against collagen Type IV, captured using a confocal scanning laser ophthalmoscope.

FIG. 4 illustrates the administration of fluorophore-tagged biomolecules into the eye via intravitreal injection for diagnostic or therapeutic purposes.

FIG. 5 shows a system diagram for ocular imaging representing the interaction between the ocular imaging device, processor, and display screen for capturing and visualizing fluorescence signals.

### DETAILED DESCRIPTION

The following detailed description provides illustrative embodiments of the present disclosure, which pertains to the field of optical imaging technologies, specifically non-invasive in vivo retinal fluorescence imaging systems for the diagnosis and management of ocular diseases. The described system leverages fluorescent-tagged biomolecules to enable precise detection of typical and pathological targets within the retina and choroid, facilitating faster and more accurate disease diagnosis and treatment. While specific examples and embodiments are described herein, these are provided for illustrative purposes only and are not to be construed as limiting the scope of the described system.

It is to be understood that certain widely recognized elements, processes, or techniques may not be described in detail to prevent unnecessary complexity in the disclosed subject matter. The described embodiments aim to include all innovative and distinguishable features and aspects of the disclosed subject matter, including various modifications, rearrangements, and combinations of components or methods that would be evident to a person of ordinary skill in the art. Consequently, the scope of the disclosed subject matter is not confined to the specific examples provided but includes all variations that align with the intent and scope of the claims.

As used herein, the term “fluorescence” means a type of photoluminescence that occurs when molecules known as fluorophores absorb electromagnetic energy, temporarily exciting them to a higher energy state, and emit light of a different, usually longer wavelength as they return to their original energy level.

As used herein, the term “fluorophore” means a molecule capable of absorbing electromagnetic energy and emitting light at a different wavelength, used in fluorescence imaging to highlight specific targets.

As used herein, the term “fluorescein” means a fluorescent dye commonly used in ophthalmology for imaging ocular blood vessels, typically excited by blue light and emitting yellow-green fluorescence.

As used herein, the term “indocyanine green (ICG)” means a fluorescent dye used in ophthalmology for imaging deeper ocular structures, excited by near-infrared light and emitting fluorescence.

As used herein, the term “confocal scanning laser ophthalmoscope (cSLO)” means an imaging device that uses visible or near-infrared light sources to scan the retina spot by spot, providing high-contrast, high-efficiency, and mydriasis-free imaging with reduced radiation exposure.

As used herein, the term “antibody” means a biomolecule produced by the immune system that specifically binds to antigens; in the context described, antibodies are conjugated to fluorophores for targeted imaging.

As used herein, the term “antibody fragment” means a portion of an antibody molecule that retains the ability to bind to specific antigens, utilized for fluorescence imaging.

As used herein, the term “intravitreal injection” means a clinical procedure involving the injection of substances directly into the vitreous humor of the eye, used to administer fluorophore-tagged biomolecules in the described technology.

As used herein, the term “macular hole” means a small break in the macula, the central part of the retina, which can affect vision and may require surgical intervention.

As used herein, the term “herpesviridae” means a family of viruses known to infect the retina, including herpes simplex virus and cytomegalovirus, which can be detected using the described technology.

As used herein, the term “fundus camera” means an imaging device used in ophthalmology to capture images of the retina, capable of fluorescence imaging with appropriate technical specifications.

As used herein, the term “pathological target” means abnormal cells, infectious agents, or other disease manifestations within the retina or choroid, identified using fluorophore-tagged biomolecules.

As used herein, the term “photobleaching” means the degradation of fluorophores due to prolonged exposure to light, which is minimized in the described system by covering the eye during imaging preparation.

As used herein, the term “diagnostic yield” means the effectiveness of a diagnostic test in identifying a disease, which is enhanced by the targeted fluorescence imaging approach described herein.

As used herein, the term “ocular diseases” means medical conditions affecting the eye, including inflammatory, infectious, and neoplastic diseases.

As used herein, the term “barrier filter” means an optical filter used in fluorescence imaging to block excitation light and allow only emitted fluorescence to reach the imaging device.

The diagnosis and management of retinal diseases have traditionally relied on fluorescence imaging techniques using dyes such as fluorescein and indocyanine green. These dyes, when injected intravenously, circulate within the ocular blood vessels and are stimulated using specific wavelengths of light to emit fluorescence, which is captured by imaging devices such as fundus cameras or confocal scanning laser ophthalmoscopes (cSLOs). While these methods have proven effective for vascular imaging and certain diagnostic applications, they are limited in their ability to detect specific pathological or anatomical targets within the retina and choroid. Furthermore, the diagnosis of inflammatory retinal conditions often necessitates invasive procedures, such as obtaining intraocular tissue samples, which are prone to false negatives due to the small sample size and low diagnostic yield. This limitation frequently results in delayed diagnoses, requiring multiple surgical interventions, as seen in conditions like intraocular lymphoma. The lack of precision and efficiency in current diagnostic methods underscores the need for a more targeted and non-invasive approach.

The present system addresses these limitations by introducing a novel retinal fluorescence imaging method that utilizes fluorophore-tagged biomolecules, such as antibodies or antibody fragments, to detect specific physiological and pathological targets within the retina and choroid. These biomolecules are conjugated to fluorophores capable of emitting light upon stimulation by electromagnetic wavelengths, which can be captured using advanced imaging devices like cSLOs. Unlike conventional approaches, this method enables precise, real-time identification of disease-specific markers, eliminating the need for invasive tissue sampling and significantly reducing diagnostic delays. For example, the system can be used to detect lymphoma cells infiltrating the retina or infectious microorganisms, such as viruses, fungi, or bacteria, with high specificity. Additionally, the method allows for the simultaneous use of multiple fluorophore-tagged biomolecules targeting different diseases, further enhancing diagnostic efficiency.

The technical solution utilizes the specific binding properties of biomolecules to pre-specified targets within the retina, combined with the enhanced sensitivity and resolution of fluorescence imaging systems. By administering these biomolecules via intraocular injection and performing imaging shortly thereafter, clinicians can obtain same-day diagnostic results, enabling timely treatment initiation. The system's capability to detect both typical anatomical components and pathological manifestations of disease represents a notable improvement over prior methods, providing a non-invasive, precise, and effective diagnostic tool for ocular diseases.

### Exemplary Systems and Methods of Use

The disclosure herein includes the use of fluorophore tagged biomolecules, such as antibodies or antibody fragments, that can detect pre-specified targets within the human retina and choroid for diagnostic and/or therapeutic purposes. These targets can be normal anatomic components of the retina or manifestations of disease. The fluorophores can be of any wavelength within the visible and non-visible electromagnetic spectrum.

The stimulation, capture, as well as amplification of the signal coming off of these fluorescent biomolecules is performed by means of confocal scanning laser ophthalmoscopy or other cameras that can stimulate the fluorophores and capture their emission.

This reaction can be performed immediately in the clinic after an intraocular injection of these fluorophore tagged biomolecules and the clinician may have same day results about the patient's diagnosis, thus initiating prompt treatment.

The biomolecules (e.g., antibodies) will be injected into the eye for the purpose of diagnosis and management of retinal diseases. This will be done in a clinic setting under local anesthesia and the patient's eye will be covered to prevent bleaching of the fluorophore. After approximately 1-2 hours imaging will be performed to investigate potential binding of the biomolecule to the target of interest. The camera to stimulate the fluorophores can be a cSLO, ideally, however a regular fundus camera can be used provided it has the necessary technical specifications for the excitation of the fluorophore. The targets of the biomolecules can be either normal anatomical components of the retina or abnormal cells or infectious agents, as a manifestation of disease.

In some embodiments of the invention, the method can include an antibody against the internal limiting membrane of the retina conjugated to a fluorophore. The internal limiting membrane is commonly peeled off the surface of the retina in cases of macular holes and complicated retinal detachments. The diagnostic test can be used to identify the extent of the prior ILM peel, which can help in the pre-operative planning of patients with recurrent macular holes.

In some embodiments of the invention, the method can include an antibody against lymphoma cells that can infiltrate the retina. The infiltration of the retina by lymphoma cells is notoriously difficult to diagnose and often requires multiple vitreous biopsies to confirm the diagnosis. With this system, the patient and clinician may have in real time the diagnosis, so there is no delay in the treatment initiation.

In some embodiments of the invention, the method can include an antibody against infectious microorganisms that are known to infect the retina. These microorganisms can be viruses of the Herpesviridae family, fungi, parasites, bacteria, among others.

In some embodiments of the invention, multiple antibodies of different targets can be injected inside the eye. The antibodies will have different fluorophores and they will be able to diagnose two different diseases.

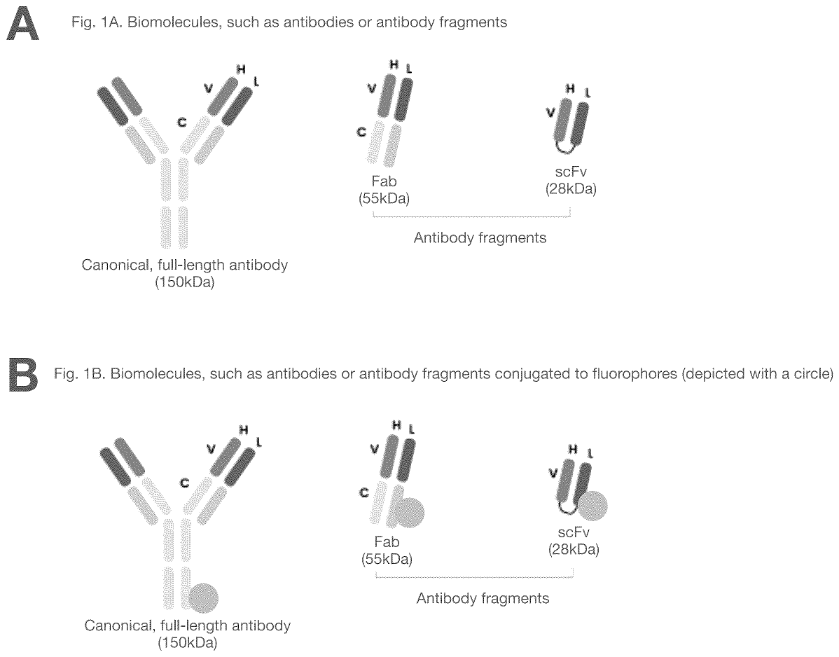

FIG. 1A illustrates example biomolecules usable with the disclosed system, including antibodies and antibody fragments, for binding to pre-specified targets within the retina or choroid.

FIG. 1B illustrates the biomolecules of FIG. 1A after conjugation to fluorophores to form fluorophore-tagged biomolecules. The fluorophores are selected to emit detectable fluorescence upon excitation, enabling in vivo imaging after intraocular administration and binding of the biomolecule to its target.

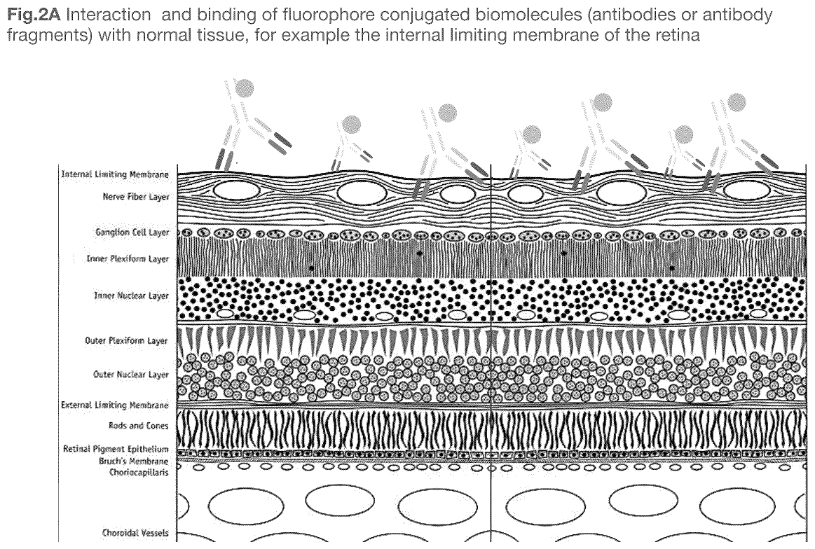

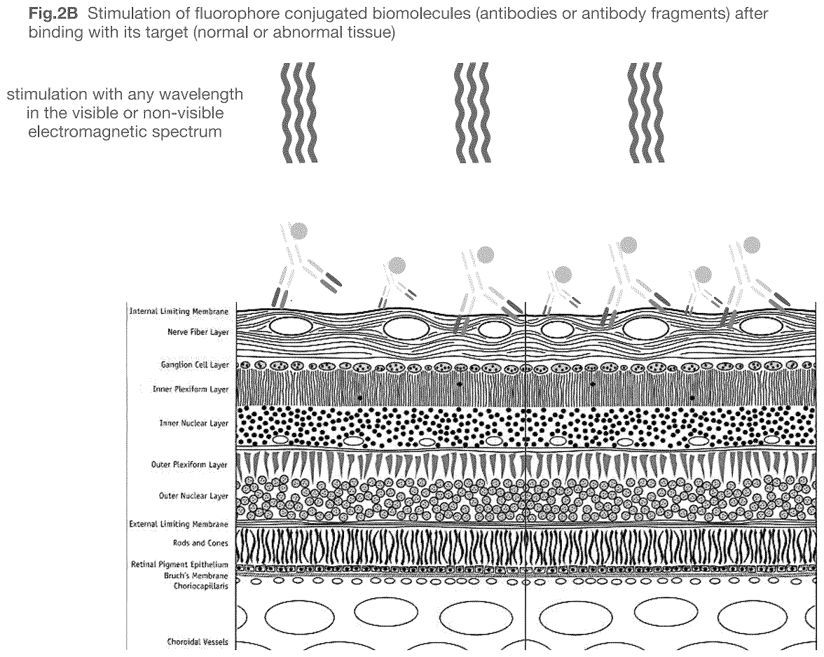

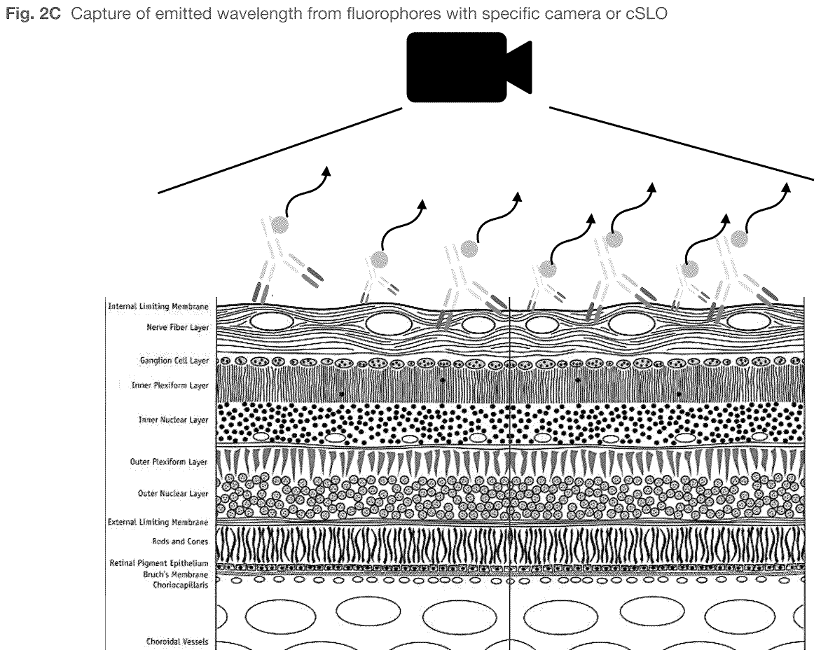

FIG. 2A illustrates an exemplary interaction in which fluorophore-conjugated biomolecules, including antibodies or antibody fragments, bind to a pre-specified target within retinal tissue. In the illustrated example, the target comprises normal tissue, such as the internal limiting membrane of the retina, and the fluorophore-conjugated biomolecules are configured to bind the target for diagnostic and/or therapeutic purposes.

FIG. 2B illustrates stimulation of the fluorophore-conjugated biomolecules after binding with the target within the retinal layers. In the illustrated embodiment, stimulation is performed using electromagnetic wavelengths selected from the visible or non-visible electromagnetic spectrum, for example using confocal scanning laser ophthalmoscopy, to excite the fluorophore associated with the bound biomolecules.

FIG. 2C illustrates capture of an emitted wavelength from the excited fluorophores by an ocular imaging device, such as a specific camera or a confocal scanning laser ophthalmoscope (cSLO). The captured emission is used for diagnostic purposes in patients with ocular diseases by enabling visualization of fluorescence associated with the bound biomolecules within the retinal layers.

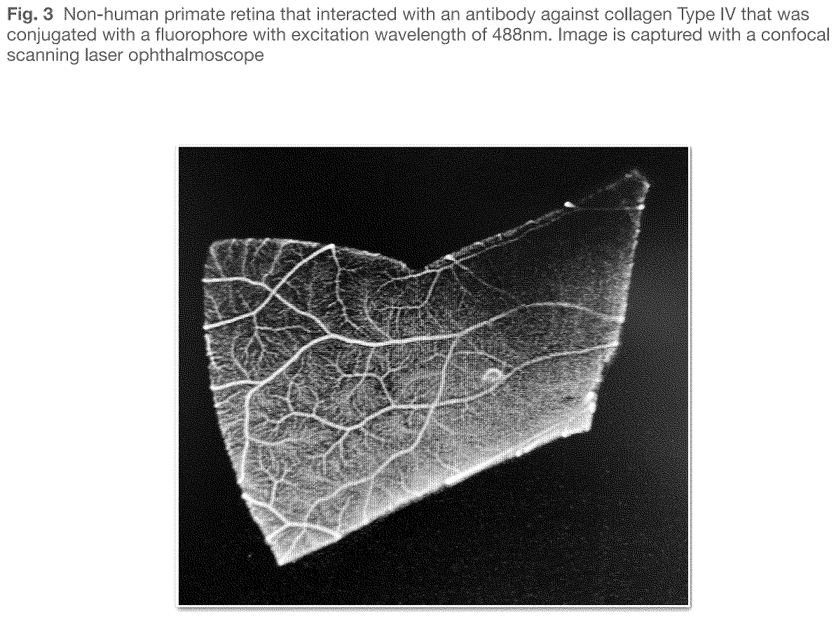

FIG. 3 depicts an exemplary microscopy image of a non-human primate retina that interacted with an antibody against collagen Type IV, wherein the antibody was conjugated with a fluorophore. In the illustrated embodiment, the fluorophore has an excitation wavelength of 488 nm, and the image is captured with a confocal scanning laser ophthalmoscope.

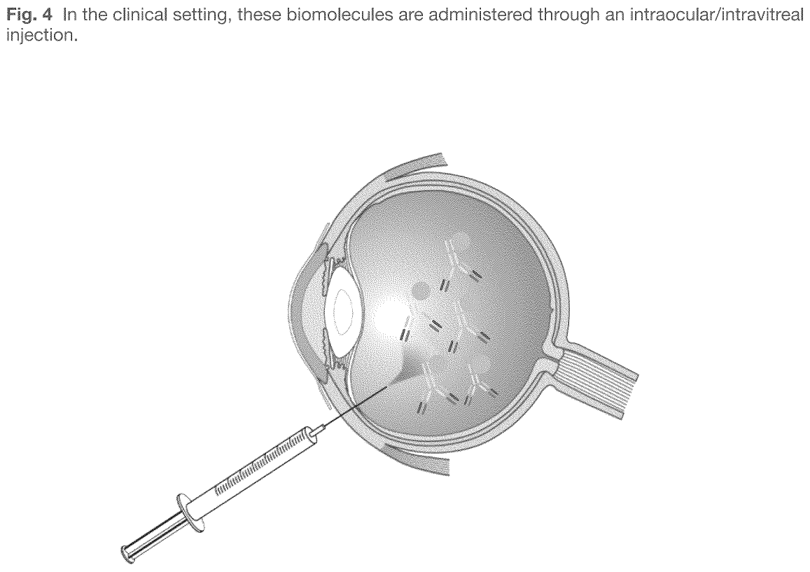

FIG. 4 illustrates an exemplary clinical administration of fluorophore-tagged biomolecules into an eye of a patient through an intraocular or intravitreal injection. In the illustrated embodiment, the injected biomolecules comprise fluorophore-tagged antibodies and/or antibody fragments configured to detect pre-specified targets within the retina or choroid for diagnostic and/or therapeutic purposes.

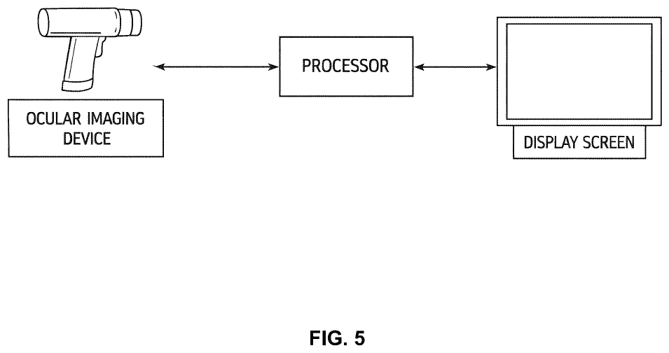

FIG. 5 shows a system diagram for ocular imaging, illustrating the interaction between primary components of the retinal fluorescence imaging system. The system comprises an ocular imaging device, a processor, and a display screen, which collectively enable the capture, processing, and visualization of fluorescence signals emitted by fluorophore-tagged biomolecules within the retina or choroid.

The processor is operatively connected to the ocular imaging device and is responsible for analyzing the captured fluorescence signals. The processor handles the data to determine the presence, distribution, or extent of the pre-specified targets within the retina or choroid. Algorithms may be employed by the processor for signal amplification, noise reduction, and image enhancement to ensure diagnostic results with precision.

Furthermore, the processor can facilitate the simultaneous analysis of multiple fluorophore-tagged biomolecules, allowing for the detection of multiple disease markers during a single imaging session. The display screen is connected to the processor and can provide a visual representation of the processed data as well as facilitate the entry of user commands through user input device. This display can allow clinicians to view the fluorescence imaging results in real time, facilitating immediate diagnosis and treatment planning. The display screen may include features for zooming, annotating, and comparing images to improve the diagnostic workflow. The visualization of fluorescence signals on the display screen supports the identification of specific disease markers, such as lymphoma cells, infectious microorganisms, or anatomical abnormalities like peeled regions of the internal limiting membrane.

The processor may be part of a system of one or more computers configured to perform particular operations or actions by virtue of having software, firmware, hardware, or a combination of them installed on the system that in operation that cause the system to perform the actions. One or more computer programs can be configured to perform particular operations or actions by virtue of including instructions that, when executed by data processing apparatus, cause the apparatus to perform the actions.

In some embodiments, the functions attributed herein to the processor are implemented, at least in part, by one or more software modules stored on a non-transitory computer-readable medium and executed by one or more processors operatively connected to the ocular imaging device. The computer-readable medium stores instructions that, when executed, cause the processor to receive image data corresponding to captured fluorescence emission from fluorophore-tagged biomolecules within a retina or choroid and to analyze the captured fluorescence signals to determine a presence, distribution, or extent of a pre-specified target.

In some embodiments, the stored instructions, when executed, cause the processor to apply one or more algorithms to the captured fluorescence signals to improve usability of the image data for diagnosis or management of ocular disease, including signal amplification, noise reduction, and image enhancement. The resulting processed data can be used to provide an output indicative of the presence, distribution, or extent of the pre-specified target as determined from the captured emission.

Furthermore, it is to be understood that the phraseology and terminology employed herein are for the purpose of description and should not be regarded as limiting. Other features, aspects, and advantages of the present invention will become apparent from the following description of the invention, taken in conjunction with the accompanying drawings, which illustrate, by way of example, various features of embodiments of the invention.

1. A method for diagnosing or managing an ocular disease in a human patient, comprising:
intraocularly administering to the patient a fluorophore-tagged biomolecule that specifically binds a pre-specified target within a retina or a choroid of the patient;
exciting the fluorophore in vivo and capturing emitted light from the biomolecule with an ocular imaging device; and
determining a presence, distribution, or extent of the target from the captured emission.

2. The method of claim 1, wherein the biomolecule comprises an antibody or an antibody fragment.

3. The method of claim 1, wherein the ocular imaging device comprises a confocal scanning laser ophthalmoscope or a fundus camera.

4. The method of claim 1, further comprising shielding the eye after the administering to reduce photobleaching.

5. The method of claim 1, wherein the administering comprises co-administering multiple biomolecules, each conjugated to a different fluorophore.

6. The method of claim 1, wherein the target comprises an internal limiting membrane of the retina and the determining comprises delineating peeled versus unpeeled regions for pre-operative planning.

7. The method of claim 1, wherein the target comprises lymphoma cells infiltrating the retina.

8. The method of claim 1, wherein the target comprises an infectious microorganism present in the retina.

9. The method of claim 1, wherein the capturing is performed approximately 1-2 hours after the administering.

10. The method of claim 1, wherein exciting the fluorophore and capturing the emitted light comprise selecting an excitation wavelength matched to an absorption band of the fluorophore and filtering detected light with at least one barrier filter configured to preferentially pass an emission band of the fluorophore while attenuating the excitation wavelength.

11. A retinal fluorescence imaging system for diagnosing or managing an ocular disease in a human patient, comprising:
at least one fluorophore-tagged biomolecule configured for intraocular administration and specific binding to a pre-specified target within a retina or a choroid of the patient;
an ocular imaging device configured to excite the fluorophore in vivo and capture emitted light from the biomolecule; and
a processor configured to determine a presence, distribution, or extent of the target from the captured emission.

12. The system of claim 11, wherein the biomolecule comprises an antibody or an antibody fragment.

13. The system of claim 11, wherein the ocular imaging device comprises a confocal scanning laser ophthalmoscope or a fundus camera.

14. The system of claim 11, further comprising a shielding element configured to cover the eye after administration to reduce photobleaching.

15. The system of claim 11, wherein the system is configured to co-administer multiple biomolecules, each conjugated to a different fluorophore.

16. The system of claim 11, wherein the target comprises an internal limiting membrane of the retina and the processor is configured to delineate peeled versus unpeeled regions for pre-operative planning.

17. The system of claim 11, wherein the target comprises lymphoma cells infiltrating the retina.

18. The system of claim 11, wherein the target comprises an infectious microorganism present in the retina.

19. The system of claim 11, wherein the ocular imaging device is configured to capture emission approximately 1-2 hours after administration.

20. A non-transitory computer-readable medium storing instructions that, when executed by one or more processors operatively connected to an ocular imaging device, cause the one or more processors to perform operations comprising:
receiving image data representing captured fluorescence emission from fluorophore-tagged biomolecules within a retina or choroid of a human patient;
analyzing the captured fluorescence emission to determine a presence, distribution, or extent of a pre-specified target within the retina or choroid; and
processing the image data using one or more algorithms comprising at least one of signal amplification, noise reduction, or image enhancement.
